SEQUENCE LISTING (20 sequences)

SEQ ID NO: 1 - DNA, 474 bp, Homo sapiens
  CDS (1)..(474): Product TNF-alpha analog Function antigen Sequence name TNF30-3
atggtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaacaactttaccgtctccttctggcttcgggtacccaaggtcagcgctagccacctcgaggtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctc

SEQ ID NO: 2 - protein, 158 aa, Homo sapiens
MVRSSSRTPSDKPVAHVVANPQAEGQLQWLNRRANALLANGVELRDNQLVVPSEGLYLIYSQVLFNNFTVSFWLRVPKVSASHLEVSYQTKVNLLSAIKSPCQRETPEGAEAKPWYEPIYLGGVFQLEKGDRLSAEINRPDYLDFAESGQVYFGIIAL

SEQ ID NO: 3 - DNA, 474 bp, Homo sapiens
  CDS (1)..(474): Product TNF-alpha analog Function antigen Sequence name TNF2-5
atggtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgacagtacattaaggccaattcgaagttcattggcatcactgagctgtctgggcaggtctactttgggatcattgccctc

SEQ ID NO: 4 - protein, 158 aa, Homo sapiens
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRQYIKANSKFIGITELSGQVYFGIIAL

SEQ ID NO: 5 - DNA, 474 bp, Homo sapiens
  CDS (1)..(474): Product TNF-alpha analog Function antigen Sequence name TNF2-3
atggtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttccagtacataaaggccaactccaagtttatcggcatcaccgagctcatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctc

SEQ ID NO: 6 - protein, 158 aa, Homo sapiens
MVRSSSRTPSDKPVAHVVANPQAEGQLQWLNRRANALLANGVELRDNQLVVPSEGLYLIYSQVLFQYIKANSKFIGITELISRIAVSYQTKVNLLSAIKSPCQRETPEGAEAKPWYEPIYLGGVFQLEKGDRLSAEINRPDYLDFAESGQVYFGIIAL

SEQ ID NO: 7 - DNA, 474 bp, Homo sapiens
  CDS (1)..(474): Product TNF-alpha analog Function antigen Sequence name TNF2-7
atggtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctccagtacatcaaagctaactccaaattcatcggcatcaccgaactggttaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctc

SEQ ID NO: 8 - protein, 158 aa, Homo sapiens
MVRSSSRTPSDKPVAHVVANPQAEGQLQWLNRRANALLANGVELRDNQLVVPSEGLYLIYSQVLFKGQGCPSTHVLQYIKANSKFIGITELVNLLSAIKSPCQRETPEGAEAKPWYEPIYLGGVFQLEKGDRLSAEINRPDYLDFAESGQVYFGIIAL

SEQ ID NO: 9 - DNA, 474 bp, Homo sapiens
  CDS (1)..(474): Product TNF-alpha analog Function antigen Sequence name TNF30-2
atggtcagatcatcttctcgaaccccgagtgacaagcctgtagcccatgttgtagcaaaccctcaagctgaggggcagctccagtggctgaaccgccgggccaatgccctcctggccaatttcaacaacttcacagttagcttctggttgagggtaccaaaggtctcggccagccacctcgagcaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctc

SEQ ID NO: 10 - protein, 158 aa, Homo sapiens
MVRSSSRTPSDKPVAHVVANPQAEGQLQWLNRRANALLANFNNFTVSFWLRVPKVSASHLEQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 11 - DNA, 474 bp, Homo sapiens
  CDS (1)..(474): Product TNF-alpha analog Function antigen Sequence name TNF30-5
atggtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgattcaacaatttcaccgtaagcttctggcttcgcgtccctaaggtgtctgcgtcgcacctcgaagggatcattgccctc

SEQ ID NO: 12 - protein, 158 aa, Homo sapiens
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRFNNFTVSFWLRVPKVSASHLEGIIAL

SEQ ID NO: 13 - DNA, 474 bp, Homo sapiens
  CDS (1)..(474): Product TNF-alpha analog Function antigen Sequence name TNF2-1
atggtcagatcatcttctcgaaccccgagtcagtacattaaagccaattctaaattcatcggtataactgagctgcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctc

SEQ ID NO: 14 - protein, 158 aa, Homo sapiens
MVRSSSRTPSQYIKANSKFIGITELQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 15 - DNA, 474 bp, Homo sapiens
  CDS (1)..(474): Product TNF-alpha analog Function antigen Sequence name TNF2-4
atggtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccccagtatatcaaggccaattcgaaattcatcggcatcacggagctcggtgaccgactcagcgctgagatcaatcggcccgactatctcgactttgccgagtctgggcaggtctactttgggatcattgccctc

SEQ ID NO: 16 - protein, 158 aa, Homo sapiens
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPQYIKANSKFIGITELGDRLSAEINRPDYLDFAESGQVYFGIIAL

SEQ ID NO: 17 - DNA, 474 bp, Homo sapiens
  CDS (1)..(474): Product TNF-alpha analog Function antigen Sequence name TNF30-1
atggtcagatcatcttctcgaaccccgagtttcaacaattttaccgtaagcttttggctccgtgtacctaaggtgtcggcctcgcacctggagcgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctc

SEQ ID NO: 18 - protein, 158 aa, Homo sapiens
MVRSSSRTPSFNNFTVSFWLRVPKVSASHLERRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 19 - DNA, 474 bp, Homo sapiens
  CDS (1)..(474): Product TNF-alpha analog Function antigen Sequence name TNF30-4
atggtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccatttaataatttcaccgtgtccttctggttgcgcgtccctaaggtaagcgcttcccacctggagaagggtgaccgactcagcgctgagatcaatcggcccgactatctcgactttgccgagtctgggcaggtctactttgggatcattgccctc

SEQ ID NO: 20 - protein, 158 aa, Homo sapiens
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPFNNFTVSFWLRVPKVSASHLEKGDRLSAEINRPDYLDFAESGQVYFGIIAL

(19) United States
(12) Patent Application Publication
(10) Pub. No.: US 2003/0185816 A1
(43) Pub. Date: Oct. 2, 2003

(54) Solubilised protein vaccines

(76) Inventors: Ole Frilev Olesen, Valby (DK); Torben Balchen, Herlev (DK); Mania Bouman, PJ Venlo (NL)

Correspondence Address: JACOBSON HOLMAN PLLC, 400 SEVENTH STREET N.W., SUITE 600, WASHINGTON, DC, 20004, US

(21) Appl. No.: 10/297,942

(22) PCT Filed: Jun. 20, 2001
(86) PCT No.: PCT/DK01/00431

(30) Foreign Application Priority Data
Jun. 21, 2000 (DK) PA2000-00966

(51) Int. Cl. A61K 39/395

(52) U.S. Cl. 424/131.1

(57) ABSTRACT

The invention relates to pharmaceutical vaccine compositions for treating or alleviating self-protein-mediated inflammatory pathologies, such as rheumatoid arthritis, Crohn's disease, enflammatory bowel disease, which vaccine compositions comprise a modified immunogenic self-protein and a surfactant capable of acting as a solubiliser. Compositions of modified human TNFalpha and cetylpyrimydium are provided.

The present invention relates to pharmaceutical vaccine compositions for preventing or treating self-protein-mediated pathologies, methods of inducing autoantibodies to self-proteins as well as methods of treatment. The invention further relates to the use of specific components in vaccine compositions.

### BACKGROUND OF THE INVENTION

It is well known that the immune system serves as a defence mechanism against invasion of the body by infectious objects such as microorganisms. Foreign proteins are effectively removed via various processes. For instance, the T helper (T_{H}) lymphocytes regulate the immune defence via a complex network of cytokines in collaboration with the Antigen Presenting Cells (APC).

T_{H} lymphocytes recognise protein antigens presented on the surface of the APC. Fragments of self-proteins are also presented by the APC. However, normally such fragments are not recognised or even ignored by the T_{H} lymphocytes. Due to this auto-antibodies are generally not found in serum.

It has, however, been recognised a dysfunction of the immune system may lead to an attack on the individual's own proteins, i.e. self-proteins or auto-proteins. Such malfunction may lead to an autoimmune disease. Also, tumour necrosis factor α (TNFα) is known to be able to cause cachexia in cancer patients and patients suffering from other chronic diseases (H. N. Langstein et al. Cancer Res. 51, 2302-2306, 1991).

The elucidation of the role of TNFα in the progression of inflammatory diseases such as rheumatoid arthritis and Crohn's Disease has resulted in the introduction of new therapies for these diseases. E.g., Immunex has developed a soluble TNFα receptor (etanercept, Enbrel™) for the treatment of rheumatoid arthritis, and Centocor has developed an anti-TNFα antibody (Infliximab, Remicade™) for the treatment of Crohn's Disease. Both these products are proteins that interact directly with the endogeneous TNFα.

In WO 95/05849 (ref. 1), an alternative approach is disclosed, for which refinements are disclosed in WO 98/46642 (ref. 2). In these documents, modified TNFα molecules are described for use in vaccines against endogeneous TNFα in the host. Because this principle does not rely on a direct interaction between the administered protein and the host's TNFα, it is likely to require a different dosing schedule. For example, it might require less protein to be administered, or it might allow longer intervals between repeat dosing. These possibilities would have clear advantages to the subject, who would receive fewer and smaller injections.

WO 95/05849 and WO 98/46642 disclose the principles involved with the vaccines, however, without fully addressing the practical problems involved in translating these principles into a workable product. Accordingly, the object of the present invention is to provide improved pharmaceutical compositions comprising modified immunogenic self-proteins, in particular modified TNFα proteins.

### SUMMARY OF THE PRESENT INVENTION

In a first aspect, the present invention relates to a pharmaceutical vaccine composition for the prevention or treatment of a self-protein-mediated pathology, which composition comprises at least one modified immunogenic self-protein and a surfactant capable of acting as a solubiliser. In other aspects, the modified self-protein is a modified human self-protein, a modified TNFα protein, or a modified human TNFα protein.

The present invention further relates to methods of inducing antibodies to a self-protein whereby the vaccine composition of the invention is administered.

In another aspect, the invention relates to method for treating self-protein-mediated pathologies.

In yet another aspects, the invention relates to the use of cetylpyridinium chloride as a component in vaccines as well as pharmaceutical vaccine compositions comprising cetylpyridinium chloride. In one embodiment, the vaccine compositions comprising cetylpridinium chloride may further comprise modified immunogenic self-protein(s).

### DETAILED DESCRIPTION OF THE PRESENT INVENTION

The present invention relates to improved pharmaceutical vaccine compositions. Such vaccine compositions of the invention comprise an active ingredient (medicament) in combination with a surfactant which is. capable of acting as a solubiliser. It was surprisingly found that the presence of such surfactant resulted in improved vaccine compositions.

Thus, in a first aspect, the present invention relates to a pharmaceutical vaccine composition for the prevention or treatment of a self-protein-mediated pathology, which vaccine composition comprises at least one modified immunogenic self-protein and, a surfactant capable of acting as a solubiliser. In particular, the self-protein may be a modified human self-protein.

In a special embodiment, the composition of the invention comprises a modified TNFα protein, in particular a modified human TNFα protein. A review of the structure and functions of the TNFα molecule is given in WO 98/46642 (ref. 2), incorporated herein by reference.

The surfactants to be used in the compositions of the invention are such which are capable of acting as a solubiliser. By this is meant that the surfactants should be capable of maintaining the modified self-protein in solution. It is well known that the formulation of a vaccine composition is important for the delivery and efficacy of the vaccine. Thus by including a surfactant as described herein, an improved vaccine composition is achieved.

It is contemplated that most of the modified self-proteins will be soluble in the presence of denaturing agents. However, this is generally incompatible with the intended use as vaccines. From the experiments performed. with the modified TNFα proteins, it was found that removal of the denaturing agents, e.g. by dialysis, generally resulted in precipatation of the protein. In the search for an alternative, it was surprisingly found that most surfactants tested were unsuitable for stabilising solutions of the modified protein in concentrations compatible with the intended use. However, it was surprisingly found that certain surfactants were capable of solubilising the proteins in concentrations compatible with the intended use. Accordingly, suitable surfactants are those which are able to maintain the proteins in solution at lower concentrations.

In one embodiment, the surfactant to be used is a cationic surfactant or a zwitterionic surfactant.

In preferred embodiments, the surfactant is selected from cetylpyridinium chloride (CPC) and Zwittergent 3-14. CPC is 1-hexadecylpyridinium chloride (CH₃(CH₂)₁₅NC₅H₅⁺Cl⁻) and Zwittergent 3-14 is 3-(N,N-demethyltetradecylammonio)propane sulphonate (CH₃(CH₂)₁₃N(CH₃)₂(CH₂)₃SO₃⁻). Another example of a suitable surfactant is benzalkonium chloride (C₁₂H₂₅N⁺(CH₃)₂CH₂C₆H₅Cl⁻).

The surfactant may suitably be used in a concentration of from 0.01% up to 2%, from 0.01% to 1.8%, from 0.01% to 1.5%, from 0.01% to 1.2%, from 0.01% to 1.0%, from 0.01% 1.5 to 0.75%, 0.01% to 0.6%, from 0.01% to 0.5%, from 0.01% to 0.2%, from 0.01% to 0.15%, from 0.01% to 0.1%, from 0.01% to 0.05%, or from 0.01 to 0.025% In a preferred embodiment, the surfactant is used in a concentration of less than 1%. In another preferred embodiment, the concentration is less than 0.1%.

The surfactant to be used in the pharmaceutical compositions of the invention is preferably CPC. CPC is preferably used in a concentration of less than 1%, in particular less than 0.1%.

The composition of the invention may further suitably comprise one or more adjuvants and/or excipients. Suitable adjuvants include aluminium hydroxide, aluminum phosphate (Adju-Phos), calcium phosphate, muramyl dipeptide analog, biodegradable microparticles and Iscom's. Suitable excipients are such which are pharmaceutically acceptable and compatible with the active ingredient. Suitable excipients are, for example, water, saline, dextrose, glycerol, ethanol, or the like, and combinations thereof. In addition, if desired, the vaccine composition may contain auxiliary substances like wetting or emulsifying agents, and/or pH buffering agents.

In one-embodiment, the adjuvant to be used is is Alhydrogel®. In particular, Alhydrogel® may be used in combination with CPC.

It further lies within the scope of the present invention to use a combination of at least two modified self-proteins in the compositions of the invention. Thus, in some embodiments, it may be advantageous to use 10, 8, 6, 5 4, 3 or 2 different modified self-proteins. In a preferred embodiment, the compositions of the invention comprises two modified self-proteins.

The vaccine composition of the invention is to be used in the treatment, alleviation or prevention of self-protein-mediated pathologies, i.e. pathologies in which the release or activity of the self-protein is involved.

Examples of such pathologies include inflammatory diseases, including rheumatoid arthritis, cancer, cachexia, multiple sclerosis, diabetes, psoriasis, osteoprosis and asthma.

In particular the disease to be prevented or treated is an inflammatory bowel disease like ulcerative colitis or Crohn's Disease as well as mixed forms thereof.

The self-protein to be modified may include any self-protein, the effects of which are not desired. Examples include, but are not limited to, TNFα, TNFI, TNFβ, interleukin 1, and K-interferon.

By the term “modified” is meant that a part of the amino acid sequence of the native self-protein has been substituted by one or more amino acids. It is contemplated that 1 to 30 amino acids are to be inserted, preferably up to 25, 20, 18, 15, 12, 10 or 5 amino acids. The substitution/insertion need not replace the same number of amino acids in the native self-protein sequence, but may replace fewer or more amino acids. Also, the substitution may be made coherently or not. The substitution may be made at any suitable region in the native self-protein, provided that the resulting modified self-protein is capable of raising neutralising antibodies against the native self-protein following administration of said modified self-protein.

In a particular embodiment, the modified self-protein is a modified human TNFα protein. When the modified self-protein is a modified TNFα protein, such may suitably be modified so as to include a substitution of a part (or parts) of the native TNFα amino acid sequence. By the modification, one or more amino acids are replaced by one or more amino acids. It is contemplated that from 1 to 30 such as up to 25, 20, 18, 15, 12, 10 or 5 amino acids are to be inserted. The insertion need not replace the same number of amino acids of the native TNFα sequence, but may be fewer or more. The important feature is that the modified TNFα protein is capable of raising neutralising antibodies against the native TNFα protein, and further that the modified protein does not show the undesired effects of the native TNFα protein, at least to a major degree.

In one embodiment, the modified TNFα protein is such selected from those having the amino acid sequence shown in SEQ ID NO.'s 2, 4, 6, 8,. 10, 12, 14, 16, 18 and 20.

In particular, the TNFα protein is modified so as to include a substitution in the front β-sheet, in any one of the connecting loops and/or in any one of the B′, I or D strands of the back β-sheet of the native human TNFα protein.

The modification is preferably such where at least one peptide fragment of the human TNFα protein has been substituted by at least one peptide known to contain an immunodominant T cell epitope or a truncated form of said protein containing an immunodominant T cell epitope and one or both flanking regions of the human TNFα protein comprising at least one TNFα B cell epitope, wherein the substitution introduces a substantial change in the amino acid sequence of any one of the strands of the front β-sheet, in any one of the connecting loops and/or in any one of the B′, I or D strands of the back β-sheet.

By the term “a substantial change” is meant a change which goes beyond a mere conservative substitution of the individual amino acids, and beyond the point mutations. In other words, the T cell epitope introduced in the TNFα sequence should preferably introduce a sequence which is of low homology with the wild-type TNFα sequence. Thus, the sequence to be inserted may have less than 50%, such as less than 30% homology with the sequence it replaces.

The modified human TNFα protein is suitably modified by the insertion of a T cell epitope which is preferably immunogenic in a majority of human HLA class II types.

The modified TNFα molecule should preferably be free from or have very low TNFα activity. By low activity is meant the activities of the native (wild-type) TNFα, in particular its cytotoxic activity as determined in the L929 bioassay (refs. 10 and 11).

In particular, the substitutions involving the following regions of the human TNFα protein are interesting:

A segment of the D strand of the back β-sheet,

at least a segment of the H strand of the front β-sheet and of the connecting loop to the I strand, preferably amino acids 132-146,

segments of the H and I strands and the entire connecting loop, preferably amino acids 132-152,

a segment of the D strand, at least a segment of the E strand and the entire connecting loop, preferably amino acids 65-79 or 64-84,

the entire C′ and C strands and a segment of the D strand, preferably amino acids 40-60, and

at least a segment of the E strand and of the front β-sheet of one or both of the connecting loops, preferably amino acids 76-90.

In accordance herewith, the modified human TNFα protein is preferably selected from proteins according to SEQ ID 2, 4, 6, 8, 10, and 12. In particular, the modified human TNFα protein may be selected from proteins according to SEQ ID 2 and SEQ ID 4.

It further lies within the scope of the presents invention to use a combination of at least two modified human TNFα proteins in the compositions of the invention. Thus, in some embodiments, it may be advantageous to use 10, 8, 6, 5, 4, or 2, preferably 2, different modified TNFα proteins. In particular, a combination of at least two of the modified proteins according to SEQ ID NO.'s 2, 4, 6, 8, 10, 12, 14, 16, 18 and 20 may be used, preferably a combination of at least two of the modified proteins according to SEQ ID NO.'s 2, 4, 6, 8, 10 and 12. In a special embodiment, a combination of the proteins SEQ ID NO. 2 and SEQ ID NO. 4 is used.

In a further aspect, the present invention relates to a method of inducing in a subject autoantibodies to a self-protein, which method comprises the administration to a subject suffering from a self-protein-mediated pathology an effective amount of a pharmaceutical vaccine composition as defined herein.

In a special aspect, the present invention relates to a method of inducing in a subject autoantibodies to TNFα which method comprises the administration to a subject suffering from a TNFα-mediated pathology an effective amount of a pharmaceutical vaccine composition as defined herein.

The subject in question is a vertebrate, such as a mammal, including a human being.

In another aspect, the present invention relates to a method for the treatment, alleviation or prevention of a self-protein-mediated pathology, which method comprises the administration to a subject in need thereof a therapeutically effective amount of a pharmaceutical vaccine composition as defined herein.

In particular, the present invention relates to a method for the treatment of a TFNα mediated pathology, in particular a human TNFα-mediated pathology, which method comprises the administration to a subject in need thereof a therapeutically effective amount of a pharmaceutical vaccine composition as defined herein.

The pathology to be treated is in particular cancer, cachexia, multiple sclerosis, diabetes, psoriasis, osteoporosis or asthma. In one embodiment, the pathology to be treated is an inflammatory bowel disease or rheumatoid arthritis. In a special embodiment, the pathology to be treated is ulcerative colitis or Crohn's Disease or a mixed form thereof.

In further aspects, the present invention relates to the use of cetylpyridinium chloride as a component in a vaccine, and to a pharmaceutical vaccine including cetylpyridinium chloride.

Such composition may in particular further comprise one or more modified immunogenic self-proteins, especially modified human proteins, like modified human TNFα proteins.

When such composition comprises one or more human TNFα proteins, they may preferably be selected from the proteins according to SEQ ID-NO.'s 2, 4, 6, 8, 10, 12, 14, 16, 18 and 20. Preferably, such human TNFα proteins may be selected from the proteins according to SEQ ID NO.'s 2, 4, 6, 8, 10, and 12. In particular, the modified human TNFα protein may be selected from the proteins according to SEQ ID No. 2 and SEQ ID No. 4.

It is to be understood. that it is equally preferred that such composition comprises two or more modified self-proteins. When such composition comprises at least two modified human TNFα proteins, such may suitably be selected from the proteins according to SEQ ID NO.'s 2, 4, 6, 8, 10, 12, 14, 16, 18 and 20, preferably from SEQ ID NO.'s 2, 4, 6, 8, 10 and 12. Especially, the proteins may be a combination of the proteins according to SEQ ID NO. 2 and SEQ ID NO. 4.

I yet a further aspect, the present invention relates to a method of immunisation of a subject, which method comprises the administration an effective amount of a pharmaceutical vaccine composition as defined herein. The subject to be immunised is a vertebrate, such as a mammal, including a human being.

In a special embodiment, the present invention relates to a method for the treatment of a human inflammatory disease, which method comprises the administration to a subject in need thereof a therapeutically effective amount of a pharmaceutical vaccine composition as defined herein.

In order to be beneficial, the modified self-protein should, apart from being capable of raising neutralising antibodies, be free from the adverse effects of the native self-protein, in particular the activity of the native self-protein should be reduced by at least 10%, at least 15%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or even 100%.

According to the invention the modified self-proteins as described herein, in particular the modified human TNFα proteins, are used in pharmaceutical vaccine compositions that comprise such modified proteins together with a surfactant capable of acting as a solubiliser. The vaccine formulation of the invention may contain other ingredients used in vaccine formulations. Strategies in formulation development of the present vaccines based on purified proteins generally correspond to formulation strategies for other protein-based drug product. Potential problems and the guidance required to overcome these problems—as for instance preservation of tertiary structure—are dealt with in several textbooks, e.g. “Therapeutic Peptides and Protein Formulation. Processing and Delivery Systems” Ed. A. K. Banga; Technomic Publishing AG, Basel 1995 (ref. 3). The use of an adjuvant, e.g., aluminium hydroxide, aluminium phosphate (Adju-Phos), calcium phosphate, muramyl dipeptide analogue, or some of the more recent developments in vaccine adjuvants such as biodegradable microparticles and Iscom's is a formulation challenge familiar to a pharmaceutical scientist working in this area.

Preparation of the vaccine compositions according to the invention which contain peptide sequences as active ingredients is generally well understood in the art, as exemplified by U.S. Pat. Nos. 4,608,251 (ref. 4), 4,601,903 (ref. 5), 4,599,231 (ref. 6), 4,599,230 (ref. 7), and 4,596,792 (ref. 8), all incorporated herein by reference. Typically, such vaccines are prepared as injectables either as liquid solutions or suspensions; solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines.

The modified self-protein is preferably in monomeric form. However, other forms such as dimers and oligomers, especially trimers and multimers, may be suited.

The vaccines are preferably administered parenterally by injection, infusion or implantation e.g. intraveneously, intramuscularly, subcutaneously, or intraarticularly. Other routes of administration e.g. oral and nasal may also be suitable.

The vaccine compositions are administered in a manner compatible with the dosage formulation, and in such amount as will be therapeutically effective and immunogenic. The quantity to be administered depends on the subject to be treated, including, e.g., the capacity of the subject's immune system to mount an immune response, and the degree of protection desired which in turn depends on the level of the self-protein in the subject. Suitable dosage ranges are expected to be of the order of several hundred micrograms active ingredient per vaccination with a preferred range from about 1 μg to 10000 μg, such as in the range from about 10 μg to 1000 μg, and especially in the range from about 50 μg to 500 μg. Suitable regimens for initial administration and booster shots are also variable but are typified by an initial administration followed by subsequent inoculations or other administrations.

As mentioned above, it may advantageous to include adjuvants in the vaccine composition. Various methods of achieving adjuvant effect for the vaccine include use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70° C. to 101° C. for 30 second to 2 minute periods respectively. DDA (dimethyldioctadecylammonium bromide), QuilA, and RIBI, MPL (monophosphoryl lipid A), and MDP (muramyl dipeptide) and its analogue have all been suggested as adjuvants. Other possibilities include aluminium hydroxide, aluminium phosphate (Adju-Phos), calcium phosphate, muramyl dipeptide analog. Some of the more recent developments in vaccine adjuvants such as biodegradable microparticles and Iscom's may also suitably be used.

In many instances, it will be necessary to have multiple administrations of the vaccine composition, usually not exceeding six vaccinations, more usually not exceeding four vaccinations and preferably one or more, usually at least about three vaccinations. The vaccinations will normally be at from two to twelve week intervals, more usually from three to five week intervals. For instance, in the treatment of autoimmune disorders, it is contemplated that 3-4 vaccinations are to be given during the first few weeks of treatment in order to get the disease into remission, optionally followed with booster vaccinations if the acute disease persists beyond the loss of antibody titers. Subsequently, booster vaccinations may be given at the onset of a relapse or flare phase.

The vaccines may be used for treating/alleviating any of the diseases described above. In particular the vaccine compositions may be used for preventing treating/-alleviating diseases, the pathophysiology of which is characterised by seIf-protein like TNFα release, in particular chronic inflammatory diseases. As examples can be mentioned rheumatoid arthritis and inflammatory bowel diseases. The latter includes ulcerative colitis and Crohn's disease as well as mixed form thereof. Other examples are cancer, cachexia, often related to cancer, multiple sclerosis, diabetes, psoriasis, osteoporosis and asthma. Cancers, which can preferably be treated or prevented according to the invention can be histogenetically classified as malignant epithelial tumours, including carcinomas and adenocarcinomas, and as malignant non-epithelial tumours, including liposarcomas, fibrosarcomas, chondrosarcomas, osteosarcomas, leiomyosarcomas, rhabdomyosarcomas, gliomas, neuroblastomas, medulloblastomas, malignant melanoma, malignant meningioma, various leukemias, various myeloproliferative disorders, various lymphomas (Hodgkin's lymphoma and non-Hodgkin lymphoma), haemangiosarcoma, Kaposi's sarcoma, lymphangiosarcoma, malignant teratoma, dysgerminoma, seminoma, and choricarcinoma.

Carcinomas and adenocarcinomas are the most abundant (accounting for approximately 90% of deaths from cancer) and are therefore interesting target diseases to treat/prevent according to the invention. The most important carcinomas and adenocarcinomas are those of the airways (espially of bronchial origin), of the breast, of the colorectum and of the stomach. However, also carcinomas and adenocarcinomas of the prostate, the ovary, of the lymphoid tissue and bone marrow, of the uterus, of the pancreas, of the esophagus, the urinary bladder, and the kidney cause a significant number of deaths and are therefore of interest.

In particular, when the modified self-protein is a modified TNFα protein, the L929 bioassay can be applied to test the immunogenic character of the modified protein (refs. 9 and 10). Antibodies raised against the modified TNFα protein in a suitable host will significantly inhibit the activity of wild-type TNFα in the L929 bioassay, and/or said antibodies will significantly inhibit the binding of wild-type TNFα to the 55 kD TNFα receptor 1 (TNFα-R55) or the to the 75 kD TNFα receptor (TNFα-R75). These suitable hosts can for instance by a primate such as a Rhesus or Cynomuleus monkey, a rodent such a rat or mouse or guinea pig, or a lagomorph such as a rabbit.

The assays suitable for evaluating the potential of the modified self-proteins are carried out using antibody or antiserum in a concentration relative to the assay setup and ought to reflect physiological conditions with respect to the involved reactants, or it ought to be possible to extrapolate to physological conditions from the results obtained from the assay. In other words, the results from the assay must indicate that a physiological concentration of antibodies against the self-protein in vivo is able to reduce the of the self-protein activity to an extent of at least 10%, 15%, 25%, 30%, 35%,. 40%, 45%, 50%, 55%, 60%, 65%, 70%, 75%, 80%, 85%, 90%, 95% or 100%. The person skilled in the art will readily know how to determine such conditions.

The modified self-proteins can be prepared according to recombinant techniques, whereby host cells (like bacteria, yeast and fungi) transformed with an expression vector for the protein are grown under suitable conditions permitting the production of the protein, and recovery of the protein. E.g. the modified TNFα proteins may be prepared by substituting the appropriate gene segments encoding peptide containing or constituting immunodominant T cell epitopes into the gene encoding the native human TNFα molecule. Subsequently the modified TNFα gene is expressed in an appropriate eukaryotic or prokaryotic expression vector. The expressed modified TNFα molecules are purified and refolded. This includes removal of denaturing agent by e.g. diafiltration. The addition of a carefully selected surfactant allows the denaturing agent to be removed without protein precipitation.

Isolated DNA molecules encoding the modified TNFα molecules have the sequences listed as SEQ ID NO.'s 1, 3, 5, 7, 9, 11, 13, 15, 17 and 19.

The vaccine composition and the modified proteins can be characterised using the following assays known to the person skilled in the art:

SDS-Page gels stained with coomassie or silver (information on size and purity),

IEF (Isoelectric focusing) (information on isoelectric point),

Endotoxin LAL assay (information on purity),

Host cell protein (information on purity),

Mass spectroscopy (information on molecular mass),

SE-HPLC with UV detection profile (information on molecule weight distribution),

N-terminal sequence (information on identity),

Circular Dichroism (information on tertiary structure),

SE-HPLC with malls detection (information on tertiary structure by light scattering),

Amino acid composition (information on identity),

Immunhgoenecity in heterologous species (mouse, rat or rabbit),

Reactivity to antibodies in Western Blots,

NMR spectroscopy,

Crystal structure,

Complete amino acid sequence determination.

The invention is further illustrated by the following examples.

### EXAMPLES

### Example 1

Preparation and Purification of Modified Human TNFα Proteins

In separate experiments, proteins according to the sequences given in SEQ ID NO. 2 (modified TNFα protein 30-3) and SEQ ID NO. 4 (modified TNFα protein 2-5) were expressed in E. coli (BL 21 cell line) according to standard techniques.

The proteins according to SEQ ID NO. 2 and SEQ ID NO. 4 were modified by the insertion (replacement) of a peptide stretch within the internal sequence of the native TNFα. The native TNFα protein has a molecular weight of 17 kDa (determined experimentally) and consists of 157 amino acids. For SEQ ID NO. 4, a peptide stretch of 15 amino acids were inserted at positions 131-146. For SEQ ID NO. 2, a peptide stretch of 21 amino acids were inserted at positions 65-84.

Inclusion bodies were collected and washed with 3 M guanidinium hydrochloride, 5 m EDTA, 1 M NaCl, 20% sucrose in 50 mM Tris buffer, pH 8.0, then solubilised overnight at 4° C. using 6 M guanidinum hydrochloride, 1 mM DTT, 5% ethanol in Tris buffer, pH 8.0. The crude proteins were purified by diafiltration through a 30 kDa membrane and then by ion exhange chromatography on an SP-Sepharose cation exchange column, eluting with 6 M urea, 20 mM Tris buffer, 10.mM DTT, pH 8.0.

Starting from 2 l of concentrated cells in each case, 179 mg of protein according to SEQ ID 2 and 48 mg of protein according to SEQ ID 4 were recovered, as determined by measuring the A₂₈₀ of the product solution. Purity was assessed by SDS PAGE. The purity was satisfactory in both cases.

### Example 2

Renaturation of Modified Human TNFα Proteins (SEQ ID NO.'s 2 and 4)

The goal was to produce a solution with a protein concentration of 1 mg/ml in a buffer containing no denaturing agents. The concentrated (5 mg/ml) protein solution from the ion exchange chromatography was diluted 10-fold into buffers containing various additives, cf. the table below. The resulting solutions were incubated at either 4° C. or room temperature. Protein precipitation was judged by eye. The results are summarised below.

| Entry | Additive | Conc. | Precipitation |
|---|---|---|---|
| A | Tween 20 | 2% | Yes |
| B | Tween 80 | 2% | Yes |
| C | Triton X-100 | 2% | Yes |
| D | Triton X-114 | 2% | Yes |
| E | Cholic acid | 2% | Yes |
| F | Octyithio- | 2% | Yes |
| | glycoside | | |
| G | Octyithioglyco- | 2% + | Yes |
| | side + sorbitol | 40% | |
| H | Brij 78 | 2% | Yes |
| I | Nega 8 | 2% | Yes |
| J | Mega 9 | 2% | Yes |
| K | Sorbitol | 40% | Yes |
| L | | 50% | Yes |
| M | | 60% | Yes |
| N | Saccharose | 40% | Yes |
| O | | 50% | Yes |
| P | | 60% | Yes |
| Q | Mannitol | 10% | Yes |
| R | | 20% | Yes |
| S | | 30% | Yes |
| T | PEG 1500 | 2% | Yes |
| U | PEG 6000 | 2% | Yes |
| V | CPC | 2% | No |
| W | | 1% | No |
| X | | 0.06% | No |
| Y | | 0.03% | Yes/No^{a)} |
| Z | Zwittergent 3-12 | 2% | Yes |
| AA | Zwittergent 3-14 | 2% | No |
| AB | | 1% | Yes |

^{a)}Some initial precipitation was observed, but this redissolved on standing at room temperature

### Examples 3-5

Vaccine Formulations of the Invention

The following experimental vaccines were prepared.

| Component | Example 3 | Example 4 | Example 5 |
|---|---|---|---|
| Protein | 400 μg | 100 μg | 400 μg |
| CPC | 0.85% | 0.21% | 0.85% |
| Alhydrogel ® | Yes | Yes | No |
| Tris | 43 mM | 11 mM | 43 mM |
| Methiolate | 0.05% | 0.05% | 0.05% |
| Vehicle | 0.01% NaCl | 0.58% NaCl | 0.09% NaCl |

In the above, protein refers to total protein, which is a 1:1 mixture of the two modified TNFα proteins of Example 1. When present, the amount of Alhydrogele® was 0.7 mg.

### Example 6

Induction of Antibodies in Animals

For the animal experiments, cynomolgus monkeys were selected. There is a 97% sequence homology between-human and cynomolgus TNFα in that only four amino acids in the TNFα sequence are not identical. Being a primate, the immune system of cynomolgus monkeys is considered to be comparable to the human immune system. Accordingly, the cynomolgus monkey is considered a relevant species for investigations of the immunogenic properties of the human TNFα autovaccine.

Groups of three cynomologus monkeys (4-12 years old, 3.1-5.4 kg) were treated with the vaccines of Examples 2-6. Each animal received 0.8 ml of vaccine by intramuscular injection into the upper leg on day 1 of the study, followed by three further injections at two-week intervals. Blood samples were taken prior to the first injection, then on day 7 and every 14 days thereafter. The total experimental period was six months.

Antibody titres in serum were determined according to GLP by an enzyme immunoassay (ELISA). Microtitre plates coated with recombinant human TNFα (rhTNFα) were incubated with appropriate diluted serum samples. After washing, an anti-monkey IgG peroxidase conjugate was added to the microtitre plates. After a second wash bound peroxidase was determined colorimetrically. The absorbance, measured at 450 nm, was correlated to an calibration curve of standard serum included on each microtitre plate. The standard serum was a pool of anti-human TNFα antibody containing sera from cynomolgus monkeys. Thus, the titre was expressed relative to a standard serum. Three dilutions, each in duplicate, were measured for each sample.

No significant clinical manifestations of toxicity were observed, indicating that the vaccinations were well tolerated.

The experiments demonstrated that repeated immunisations with the modified TNFα formulations induced a reversible anti-human TNFα antibody response in cynomolgus monkeys. Increases in antibody titre were evident three weeks after the first immunisation. Antibody titre reached a maximum between days 35 and 49 (after the third or fourth immunisation) of the study. In most animals the antibody titre declined after the third or fourth immunisation with a half-life of about three weeks. This is very close to the half-life of IgG1 which is most prevalent in serum suggesting that the production of antibodies stops when the immunisations are discontinued. At the end of the experimental period, the antibody titres had returned to the pre-immunised levels in about half of the animals.

The mean increase in antibody titre is summarised below.

| Group | Vaccine | Increase in titre |
|---|---|---|
| 1 | Example 3 | 136-fold |
| 2 | Example 4 | 29-fold |
| 3 | Example 5 | 132-fold |

A control group of animals was treated with a vaccine that included both Alhydroglel® and 400 μg of protein, but no CPC. Peak antibody titre was reached after 49-63 days, and the mean increase in titre was 65-fold.

These results show that CPC is well tolerated as a component in a vaccine formulation, and that it can behave as an adjuvant. Comparing groups 1 and 2 shows that the antibody response is dose dependent, confirming that this effect is a consequence of the experiment. Groups 1 and 3 received equal doses of protein, and group 3 did not receive the adjuvant (Alhydrogele). Contrary to expectations, the antibody response is very similar in each group. Now comparing group 1 with the control group, which received the same dose of protein and adjuvant, but no CPC, it is seen that group 1 responded twice as much as the control group. Accordingly, CPC acts as an adjuvant. It appears that CPC has two benefits in this system, neither of which could be predicted. Firstly, it is capable of solubilising the protein at concentrations sufficiently low to be compatible with administration to patients. Secondly, it increases the immunogenicity of the vaccine.

References

1. WO 95/05849 (Mouritsen et al.)

2. WO 98/46642 (Jensen et al.)

3. “Therapeutic Peptides and Protein Formulation. Processing and Delivery Systems” Ed. A. K. Banga; Technomic Publishing AG, Basel 1995

4. U.S. Pat. No. 4,608,251

5. U.S. Pat. No. 4,601,903

6. U.S. Pat. No. 4,599,231

7. U.S. Pat. No. 4,599,230

8. U.S. Pat. No. 4,596,792

9. Abstract from H Hay, and J Cohen, J. Clin. Lab. Immunol. 29(3), 151-155-(1989)

10. Abstract from D R Branch, A Shah, and L J Guilbert, J. Immunol. Methods. 143(2), 251-241 (1991)

1. A pharmaceutical vaccine composition for the prevention or treatment of a self-protein-mediated pathology comprising at least one modified immunogenic self-protein and a surfactant capable of acting as a solubiliser.

2. The composition according to claim 1, wherein the self-protein is a modified human self-protein.

3. The composition according to claim 1, wherein the self-protein is a modified TNFα protein.

4. The composition according to claim 3, wherein the TNFα protein is a human TNFα protein.

5. The composition according to claims 1-4, wherein the surfactant is a cationic surfactant.

6. The composition according to claims 1-4, wherein the surfactant is selected from cetylpyridinium chloride and Zwittergent 3-14.

7. The composition according to claim 6, wherein the concentration of the surfactant is less than 1%.

8. The composition according to claim 6, wherein the concentration of the surfactant is less than 0.1%.

9. The composition according to claims 1-5, wherein the surfactant is cetylpyridinium chloride.

10. The composition according to claim 9, wherein the concentration of centylpyridinium chloride is less than 1%.

11. The composition according to claim 9, wherein the concentration of cetylpyridinium chloride is less than 0.1%.

12. The composition according to any one of claims 1-11 further comprising one or more adjuvants and/or excipients.

13. The composition according to claim 12, wherein the advjuvant is selected from aluminium hydroxide, aluminium phosphate (Adju-Phos), calcium phosphate, muramyl dipeptide analog, biodegradable micropartidles and Iscom's.

14. The composition according to claim 12, wherein the adjuvant is Alhydrogel®.

15. The composition according to any one of claims 1-14, wherein pathology is an inflammatory disease or rheumatoid arthritis.

16. The composition according to claim 15, wherein the disease is an inflammatory bowel disease.

17. The composition according to claim 16, wherein the inflammatory bowel disease is ulcerative colitis or Crohn's Disease.

18. The composition according to claims 1-14, wherein the disease is cancer, cachexia, multiple sclerosis, diabetes, psoriasis, osteoporosis or asthma.

19. The composition according to any one of claims 1-18, wherein the modified human TNFα protein is modified so as to include a substitution in the front β-sheet, in any one of the connecting loops and/or in any one of the B′, I or D strands of the back β-sheet of the native human TNFα protein.

20. The composition according to claim 17, wherein the modified human TNFα protein is modified so as to include a T cell epitope which is immunogenic in a majority of human HLA class II.

21. The composition according to claim 4, 19 or 20, wherein the modified human TNFα protein is selected from proteins according to SEQ ID NO.'s 2, 4, 6, 8, 10, 12, 14, 16, 18 and 20.

22. The composition according to claims 19-21, wherein the modified human TNFα protein is selected from proteins according to SEQ ID NO.'s 2, 4, 6, 8, 10 and 12.

23. The composition according to claims 19-21, wherein the modified human TNFα protein is selected from proteins according to SEQ ID NO. 2 and SEQ ID NO. 4.

24. The composition according to claim 4 or 19, which comprises at least two human TNFα proteins.

25. The composition according to claim 24, wherein each modified humnan TNFα protein is independently modified so as to include a substitution in the front β-sheet, in any one of the connecting loops and/or in any one of the B′, I or D strands of the back β-sheet of the native human TNFα protein.

26. The composition according to claim 24 or 25, wherein the two modified human TNFα proteins are selected from proteins according to SEQ ID NO.'s 2, 4, 6, 8, 10, 12, 14, 16, 18 and 20.

27. The composition according to claim 24 or 25, wherein the two modified human TNFα proteins are selected from proteins according to SEQ ID NO.'s 2, 4, 6, 8, 10 and 12.

28. The composition according to claim 24 or 25, wherein the two modified human TNFα proteins are proteins according to SEQ ID NO. 2 and SEQ ID No 4.

29. A method of inducing in a human subject autoantibodies to a self-protein, which comprises the administration to a subject suffering from a self-protein-mediated pathology an effective amount of a pharmaceutical vaccine composition according to claims 1-28.

30. A method of inducing in a human subject autoantibodies to TNFα which comprises the administration to a subject suffering from a TNFα-mediated pathology an effective amount of a pharmaceutical vaccine composition according to any one of claim 1-28.

31. A method for the treatment of a self-protein-mediated pathology, which comprises the administration to a subject in need thereof a therapeutically effective amount of a pharmaceutical vaccine composition according to claims 1-28.

32. A method for the treatment of a human TFNα-mediated pathology, which comprises the administration to a subject in need thereof a therapeutically effective amount of a pharmaceutical vaccine composition according to any one of claims 1-28.

33. The method according to claim 32, wherein the pathology is an inflammatory disease.

34. The method according to claim 33, wherein the inflammatory disease is an inflammatory bowel disease or rheumatoid arthritis.

35. The method according to claim 34, wherein the inflammatory bowel disease is ulcerative colitis or Crohn's Disease.

36. The method according to claim 31 or 32, wherein the pathology is cancer, cachexia, multiple sclerosis, diabetes, psoriasis, osteoprosis or asthma.

37. Use of cetylpyridinium chloride as a component in a vaccine.

38. A pharmaceutical vaccine composition which includes cetylpyridinium chloride.

39. The composition according to claim 38, which further comprises one or more modified immunogenic proteins.

40. The composition according to claim 39, wherein the immunogenic protein is a modified human protein.

41. The composition according to claims 29-40, wherein the immunogenic-protein is a modified human TNFα protein.

42. The composition according to claim 41, wherein the human TNFα protein is selected from the proteins according to SEQ ID NO.'s 2, 4, 6, 8, 10, 12, 14, 16, 18 and 20.

43. The composition according to claim 41, wherein the human TNFα protein is selected from the proteins according to SEQ ID NO.'s 2, 4, 6, 8, 10 and 12.

44. The composition according to claim 41, wherein the human TNFα protein is selected from the proteins according to SEQ ID NO. 2 and SEQ ID NO. 4.

45. The composition according to claim 39, wherein the TNFα protein is a combination of the proteins according to SEQ ID NO. 2 and SEQ ID NO. 4.

46. A method of immunisation of a human subject which comprises the administration an effective amount of a pharmaceutical vaccine composition according to any one of claims 38-45.

47. A method for the treatment of a human inflammatory disease which comprises the administration to a subject in need thereof a therapeutically effective amount of a pharmaceutical vaccine composition according to any one of claims 38-45.
